SEQUENCE LISTING (17 sequences)

SEQ ID NO: 1 - protein, 9 aa, Artificial Sequence
  Description of Artificial Sequence Synthetic peptide
  MOD_RES (5)..(5): phospho-Thr
LLCSTPNGL

SEQ ID NO: 2 - protein, 9 aa, Artificial Sequence
  Description of Artificial Sequence Synthetic peptide
LLCSTPNGL

SEQ ID NO: 3 - protein, 9 aa, Artificial Sequence
  Description of Artificial Sequence Synthetic peptide
  MOD_RES (5)..(5): phospho-Thr
PLHSTPNGL

SEQ ID NO: 4 - protein, 7 aa, Artificial Sequence
  Description of Artificial Sequence Synthetic peptide
  MOD_RES (5)..(5): phospho-Thr
PLHSTAI

SEQ ID NO: 5 - protein, 6 aa, Artificial Sequence
  Description of Artificial Sequence Synthetic peptide
  MOD_RES (4)..(4): phospho-Thr
LHSTAI

SEQ ID NO: 6 - protein, 6 aa, Artificial Sequence
  Description of Artificial Sequence Synthetic peptide
  MOD_RES (5)..(5): phospho-Thr
PLHSTA

SEQ ID NO: 7 - protein, 11 aa, Artificial Sequence
  Description of Artificial Sequence Synthetic peptide
  MOD_RES (7)..(7): phospho-Thr
GPLATSTPKNG

SEQ ID NO: 8 - protein, 11 aa, Artificial Sequence
  Description of Artificial Sequence Synthetic peptide
  MOD_RES (7)..(7): phospho-Thr
GPLATSTPNGL

SEQ ID NO: 9 - protein, 9 aa, Artificial Sequence
  Description of Artificial Sequence Synthetic peptide
  MOD_RES (5)..(5): phospho-Thr
PLHSTPKNG

SEQ ID NO: 10 - protein, 7 aa, Artificial Sequence
  Description of Artificial Sequence Synthetic peptide
  MOD_RES (5)..(5): phospho-Thr
PLHSTAI

SEQ ID NO: 11 - protein, 6 aa, Artificial Sequence
  Description of Artificial Sequence Synthetic peptide
  MOD_RES (2)..(2): phospho-Thr
STPNGL

SEQ ID NO: 12 - protein, 3 aa, Artificial Sequence
  Description of Artificial Sequence Synthetic peptide
  MOD_RES (2)..(2): phospho-Thr
STA

SEQ ID NO: 13 - protein, 4 aa, Artificial Sequence
  Description of Artificial Sequence Synthetic peptide
  MOD_RES (2)..(2): phospho-Thr
STAI

SEQ ID NO: 14 - protein, 4 aa, Artificial Sequence
  Description of Artificial Sequence Synthetic peptide
  MOD_RES (2)..(2): phospho-Thr
STPL

SEQ ID NO: 15 - protein, 4 aa, Artificial Sequence
  Description of Artificial Sequence Synthetic peptide
PNGL

SEQ ID NO: 16 - protein, 4 aa, Artificial Sequence
  Description of Artificial Sequence Synthetic peptide
PKNG

SEQ ID NO: 17 - protein, 5 aa, Artificial Sequence
  Description of Artificial Sequence Synthetic peptide
GPLAT

(19) United States
(12) Patent Application Publication
(10) Pub. No.: US 2019/0269784 A1
(43) Pub. Date: Sep. 5, 2019

(54) Selective Inhibitors of the Polo-Like Kinase 1 Polo-Box Domain

(71) Applicant: UNIVERSITY OF SOUTH CAROLINA, Columbia, SC (US)

(72) Inventor: Campbell McInnes, Irmo, SC (US)

(21) Appl. No.: 16/245,652

(22) Filed: Jan. 11, 2019

Related U.S. Application Data
(60) Provisional application No. 62/616,161, filed on Jan. 11, 2018.

(51) Int. Cl. A61K 47/64; A61K 31/166; A61P 35/00

(52) U.S. Cl. CPC A61K 47/64; A61P 35/00; A61K 31/166

(57) ABSTRACT

Inhibitors that are specific for the PBD domain of the PLK1 protein are described. The inhibitors include fragment ligated inhibitors that include one or more amino acids of a starting peptide upon which the inhibitors are based and also include non-peptidic inhibitors. The inhibitors include a benzoic acid-based derivative that mimics the structure activity relationship of amino acid residues of known peptide inhibitors. The inhibitors exhibit high selectivity for the PLK1 isotype.

### CROSS REFERENCE TO RELATED APPLICATION

This application claims filing benefit of U.S. Provisional Patent Application Ser. No. 62/616,161 entitled “Selective Inhibition of the polo-Like Kinase 1 PBD For Cancer Therapy,” having a filing date of Jan. 11, 2018, which is incorporated herein by reference in its entirety.

### BACKGROUND

The family of polo-like kinase (PLK) proteins are central players in regulating entry into and progression through mitosis. The four known human PLKs have non-redundant and non-overlapping functions. A significant body of literature has validated PLKs as anti-tumor drug targets and suggested that profound anti-proliferative activity is achieved through selective inhibition of PLK1 functions. Over-expression of PLK1 is frequently observed and PLK1 expression is a prognostic indicator for outcome of patients suffering from various tumors. For example, more than half of prostate cancers over-express PLK1 and this expression is positively correlated with tumor grade. PLK1 is also extensively over-expressed in colorectal cancer and has been demonstrated to be a potential therapeutic target in colorectal cancer cell lines with inactivated p53. Moreover, it has been reported that p53 transcriptionally regulates PLK1 expression, providing more direct evidence that PLK1 is oncogenic when p53 is mutated. Thus, there is a strong rationale for pursuing PLK1 as an anti-tumor drug target. Indeed, the therapeutic rationale for PLK inhibition has been validated through studies with PLK1-specific antisense oligonucleotides and shown to profoundly induce growth inhibition in cancer cells both in vitro and in vivo.

PLK1 is comprised of two structural domains: the kinase domain, containing the ATP binding site; and the protein substrate recognition polo-box domain (PBD). The PBD is a phosphopeptide binding region of the protein that determines substrate recognition and subcellular localization and as such is critical to numerous roles in regulating mitosis. Although several ATP-binding site inhibitors of PLK1 have advanced to clinical trials, there is concern about the selectivity of these compounds for PLK isoforms. This is problematic because PLK3 has been shown to act as a tumor suppressor, and in fact might have opposing functions to PLK1. Moreover, a single mutation in PLK1 (Cys67Val) confers substantial resistance to several structurally unrelated ATP-binding site inhibitors. An alternative approach to developing potent and selective PLK1 inhibitors is to target the PBD.

What are needed in the art are potent and highly selective PLK1 inhibitors that incorporate non-peptidic segments that exhibit equivalent or improved structure activity relationship to known inhibitor peptides. Such non-peptidic PLK1 inhibitor segments that target the distinctive binding site on the PBD can be used to generate novel anticancer therapeutics that are both discriminatory to PLK1 and less toxic to normal cells.

### SUMMARY

According to one embodiment, disclosed are inhibitors that target the PBD of PLK1. Disclosed inhibitors have been developed from PBD-interacting peptides according to an approach in which one or more amino acid residues of a starting peptide has been replaced with a benzoic acid-based fragment. The benzoic acid-based segments of the inhibitors can include long chain alkyl derivatives (e.g., C6 or higher) and phenyl alkoxy derivatives, among others.

In one embodiment, the starting PBD-interacting peptide that is modified to include a benzoic acid-based fragment includes LLCS[pT]PNGL (SEQ ID NO: 1), which is a Cdc25C PBD substrate peptide. In another embodiment, the peptide that is modified to include a benzoic acid-based fragment includes the PBD-interacting peptide (PBIP) PLHS[pT]AI (SEQ ID NO:10). In one embodiment, disclosed inhibitors can include a benzoic acid-based fragment as a replacement for the amino acid residues upstream of a phosphorylated threonine of a starting peptide.

Also disclosed are methods of utilizing the inhibitors. For instance, a method can include locating an inhibitor that includes a benzoic acid-based fragment as described herein an area that includes a cell that expresses PLK1 (e.g., a prostate cancer cell) and thereby inhibiting proliferation of the cell.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates a method utilized in forming disclosed inhibitors.

FIG. 2 illustrates a structural model describing interaction of a derivatized inhibitor as described herein with the hydrophobic PBD groove of PLK 1.

FIG. 3 illustrates another structural model describing interaction of a derivatized inhibitor as described herein with the hydrophobic PBD groove of PLK 1.

FIG. 4 illustrates interactions of a starting peptide (SEQ ID NO: 2) with the PBD of both PLK1 (3BZI) and PLK3 (homology model).

FIG. 5 illustrates cell viability of Hela cells incubated in the presence of inhibitors as described herein.

FIG. 6 illustrates cell viability of prostate cancer (PC3) cells incubated in the presence of inhibitors as described herein.

FIG. 7 Cell cycle analysis of PC3 cells following 24 hour treatment with an inhibitor as described herein.

FIG. 8 illustrates the sensitivity and resistance of wild type (WT) and mutant (C67V) RPE cells, respectively, to inhibitors as described herein.

FIG. 9 presents immunoblotting results for cells treated with an inhibitor as disclosed herein and subjected to anti-BubR₁, anti-β tubulin, and anti-pHH3. As indicated, the examined inhibitor blocked the cells at mitosis and inhibited BubR₁ phosphorylation.

### DETAILED DESCRIPTION

The following description and other modifications and variations to the present invention may be practiced by those of ordinary skill in the art, without departing from the spirit and scope of the present invention. In addition, it should be understood that aspects of the various embodiments may be interchanged both in whole and in part. Furthermore, those of ordinary skill in the art will appreciate that the following description is by way of example only, and is not intended to limit the invention.

In general, disclosed herein are inhibitors that are specific for the PBD domain of the PLK1 protein. In one embodiment, the inhibitors can be fragment ligated inhibitors that include one or more amino acids of a starting peptide upon which the inhibitors are based. In other embodiments, the inhibitors can be non-peptidic, in which case all of the amino acid residues of a starting peptide have been replaced with non-peptidic fragments, each fragment replacing one or more amino acid residues of the starting peptide.

The non-peptide fragment(s) of an inhibitor can substantially maintain and mimic the structure activity relationship of the replaced peptide segment of the peptide inhibitor upon which the new inhibitor is based. As such, disclosed inhibitors can be better suited for use in clinical settings as compared to previously known peptide inhibitors that generally exhibit poor pharmacokinetic properties. For instance, a non-peptide fragment ligated to the terminal portion of an inhibitor can protect any core amino acid residues remaining in the inhibitor from proteolytic degradation and thereby improve the half-life of the inhibitor within a cell as compared to a fully peptidic inhibitor.

Disclosed inhibitors and non-peptidic fragments thereof can exhibit comparable or improved affinity to the PBD as compared to known peptide PBD inhibitors and can possess anti-proliferative phenotypes in cells. The inhibitors can show promise as isotype, kinase selective, non-ATP competitive inhibitors and can be effective against cells that are resistant to ATP inhibitors. As such, disclosed inhibitors and non-peptide fragments thereof can provide impetus for the development of PLK1 selective anti-tumor therapeutics that can expand treatments to tumors resistant to current standard therapeutics. Moreover, disclosed inhibitors can discriminate between PLK family members and can specifically target the PBD of PLK1 and as such can avoid unintended consequences due to opposing functions of different members of the PLK family. In addition, cell cycle analysis as described further herein shows that disclosed inhibitors can induce a profound G2/M block and as such can phenocopy PLK1 knockdowns.

The non-peptide fragments of the disclosed inhibitors have been developed via examination of protein-protein interactions between known inhibitors and the PBD of PLK1. An iterative approach as has been described previously (see, e.g., U.S. Pat. Nos. 9,175,357; 9,376,465; 9,982,015; and 10,067,311; all of which being incorporated herein by reference) has been applied to develop benzoic acid-based fragment alternatives for portions of peptide inhibitors.

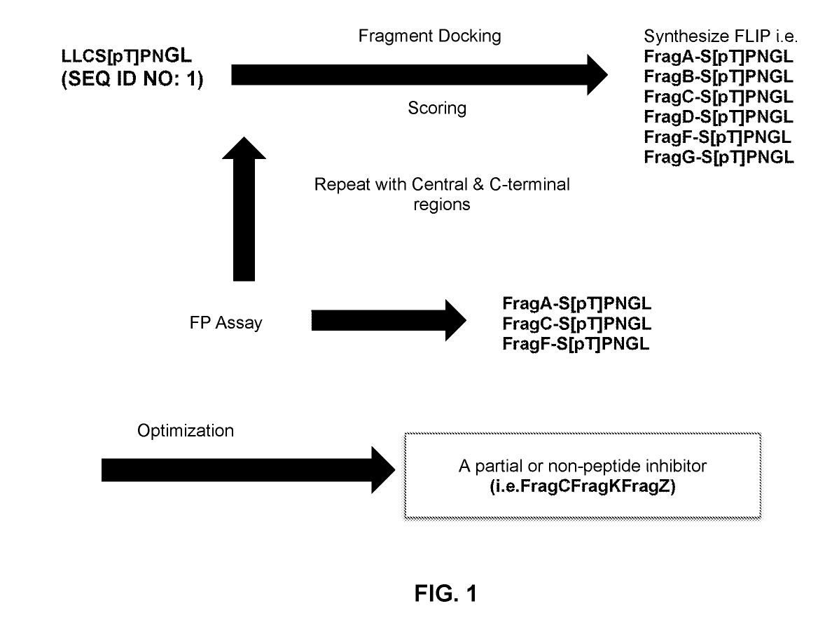

FIG. 1 is a flow diagram schematically illustrating a method for developing an inhibitor as described herein. According to the process, a starting peptide is selected that is a known inhibitor, e.g., a known PBD peptide inhibitor. By way of example, the Cdc25C PBD substrate peptide LLCS[pT]PNGL (SEQ ID NO: 1) can be utilized, as illustrated in FIG. 1. The structure activity relationship (SAR) can be determined for the starting peptide inhibitor as can be the 3-D structure for the inhibitor in complex with a PBD, e.g., a PLK1 PBD. A substitute benzoic acid-based fragment (e.g., FragA, FragB, etc.) can then be docked and scored with regard to affinity of the new FLIP that incorporates the fragment with the remainder of the starting peptide with the PBD. Reiteration and optimization of the fragment can be carried out to iteratively develop a plurality of FLIPs, as shown, and determine a best fit fragment replacement segment for the truncated peptidic fragment. The process can then be repeated for the remainder of the original peptide inhibitor, e.g., for a central region of the inhibitor and a C-terminal region of the inhibitor. An assay, such as a fluorescence polarization (FP) assay can be utilized, in which binding of a fluorescent tracer peptide to a protein increases polarization of the mitted fluorescence. Such an assay can be utilized to optimize each fragment for each region of a starting peptide. Upon fragment optimization for each region of the starting peptide, a final product, which can be a partial peptidic or a completely non-peptidic inhibitor, as shown, can be formed.

For instance, in one embodiment, a method can include replacement of the N-terminal hydrophobic motif in a CDC25c PBD substrate peptide (e.g., SEQ ID NO: 1) with a non-peptide fragment that can provide similar structure activity relationships as the replaced peptide fragment. This approach can be informed by peptide structure activity data obtained through synthesis and testing of truncated and mutated analogs of known PBD binding motifs. Any testing methodology as is known in the art can be utilized in determination of the structure activity relationship of the peptide inhibitor utilized as a basis. For instance, analog formation, computational design, comparative binding assays, and the like can be utilized according to known practice to determine the structure activity relationships of the peptide inhibitor, and particularly in determining the structure activity relationship of one or more of the terminal amino acid residues and/or the core amino acid residues of the peptide inhibitor that will be replaced by a non-peptide fragment in formation of a fragment ligated inhibitor.

While most of the present application is directed to benzoic acid-based fragments as replacement for or ligated to the N-terminus of a starting peptide, it should be understood that disclosed fragments can replace one or more terminal amino acid residues, e.g., one, two, three, four, or more terminal amino acid residues, of a starting peptide at one or both of the N-terminal and the C-terminal of the peptide used as a starting peptide. Moreover, disclosed fragments can alternatively be utilized in a central region of a starting peptide, for instance in forming a completely non-peptidic inhibitor.

Peptide inhibitors that can be utilized as the basis for development of the fragment ligated inhibitors can include any peptide inhibitor capable of selectively inhibiting PLK1. For instance, the starting peptide inhibitor can include both native peptides and variants thereof. In one embodiment, the starting peptide inhibitor can include a core group of amino acid residues (e.g., about three or more amino acid residues) that will also form a core peptide of a FLIP that includes a benzoic acid-based fragment as described. For example, a core peptide of amino acid residues that is present in a known protein inhibitor and that is utilized as a basis for formation of an inhibitor as described can correlate to a core peptide of the same amino acid residues in the final inhibitor.

In certain embodiments, the starting peptide upon which an inhibitor is based can include the Cdc25C peptide LLCS[pT]PNGL (SEQ ID NO: 1). In another embodiment, the starting peptide can include the PBIP PLHS[pT]AI (SEQ ID NO:10). FLIPs based upon these starting peptides can include peptide fragments of the starting peptides or modifications thereof, examples of which include, without limitation, S[pT]PNGL (SEQ ID NO: 11), S[pT]A (SEQ ID NO: 12), S[pT]AI (SEQ ID NO: 13), and S[pT]PL (SEQ ID NO: 14)). The peptide fragment can be ligated directly to the benzoic acid-based fragment at the N-terminus and/or at the C-terminus in the formation of FLIPs.

Through examination of PLK1vs PLK3 PBD binding as described in the Examples section below, information on the determinants of selectivity can be revealed and utilized for development of inhibitors including generation of both FLIP molecules and in further development of non-peptidic drug-like small molecules.

In one embodiment, inhibitors can include a benzoic acid-based fragment ligated to a core peptide, forming a FLIP. In other embodiments, a non-peptidic inhibitor can include a benzoic-acid based fragment bonded to one or more additional non-peptidic fragments.

Benzoic acid-based fragments of disclosed inhibitors can have the following general structure:

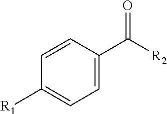

In which R₁ is an alkyl group, an alkoxy group, an alkylthio group, an alkylamino group, or a phenyl alkoxy group, and R₂ is a peptide or nonpeptidic fragment, e.g., a peptide fragment selected from SEQ ID NO: 11-14. As illustrated, the derivatization of the benzoic acid group can be a para-substitution (i.e., a 4-group on the benzoic acid), but this is not a requirement and in other embodiments, the derivatization of the benzoic acid group can include an ortho- and/or meta-substitution, optionally in conjunction with a para-substitution.

In one embodiment, R₁ is a long chain alkyl group, and in one particular embodiment a C6 or longer, or a C8 or longer alkyl chain. For example, in particular embodiments, the benzoic acid derivative can be a 4-octyl benzoic acid or a 4-nonyl benzoic acid.

In another embodiment, R₁ can be a phenyl alkoxy group, e.g., a phenyl methoxy or a phenyl ethyoxy that is linked to the benzoic acid of the derivatization via the alkoxy group. Examples of such derivative include, without limitation,

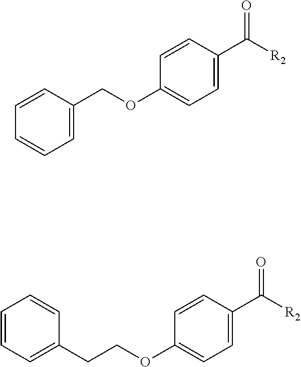

Optionally, a phenyl alkoxy group can include further substitution, for instance halogen substitution on the phenyl. One example of such a benzoic acid-based fragment is:

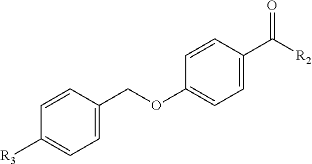

In which R₃ is halogen, e.g., F, Cl, Br, I.

Disclosed inhibitors can be drug-like small molecule PBD-inhibitors that have a high binding affinity for PLK1 while retaining selectivity against other PLKs including PLK3, a known tumor suppressor. For instance, the disclosed inhibitors can have a selectivity index for PLK 1 over PLK 3 of about 100 or more, for instance about 200 or more, about 500 or more or about 1000 or more in some embodiments, as utilized herein, and as described further in the examples section below, the term “selectivity index” is generally defined as a preference for an inhibitor for a first substrate as compared to a second substrate. For instance, the selectivity index can be the ratio of IC₅₀ value of an inhibitor for PLK1 PBD to the IC₅₀ value of the same inhibitor for PLK3 PBD. The selectivity index can also take into account the difference in affinity of a control agent, e.g., a fluorescent tracer for the substrate, e.g., the targeted PBD (either PLK1 PBD or PLK3 PBD). As described further in the examples section below, in one particular embodiment, this affinity difference for a control agent is 5 fold greater for PLK1 PBD than PLK3 PBD. In this particular embodiment, the selectivity index would thus be (IC₅₀ PLK3 PBD/IC₅₀ PLK1 PBD)×5.

The benzoic acid-based fragments of the disclosed inhibitors provide successful mimicry of critical determinants of the PBD motif and thus provide a drug-like molecular fragment that can replace peptide residues of known inhibitors, and in particular N-terminal residues. SAR and molecular modeling studies in both the peptide and FLIP contexts described further herein demonstrate that the both the N and C-termini contribute to the high selectivity of known peptide inhibitors (e.g., PBIP and Cdc25C peptide inhibitors) and that truncated compounds lose both potency and selectivity for PLK1. Beneficially, FLIPs and non-peptidic inhibitors that include the disclosed benzoic acid-based derivatives as replacement for residues of the previously known peptide inhibitors can exhibit anti-proliferative activity and drug resistance as well as high selectivity for PLK1.

The present disclosure may be better understood with reference to the Examples provided below.

### EXAMPLE 1

To establish structure-activity relationships for PBD binding sequences, a peptide library as shown in Table 2, below, was designed to probe the contributions of the N- and C-terminal residues of the recognition sequences from Cdc25C (LLCS[pT]PNGL (SEQ ID NO: 1)) and PBIP (PLHS[pT]AI (SEQ ID NO:10)) in a systematic fashion and these compounds were tested in an fluorescent polarization assay to quantify competitive binding of the phosphopeptides to the PBD domain of PLK1.

According to the assay, binding of a fluorescent tracer peptide to a protein increased polarization of the emitted fluorescence. Fluorescence polarization, in millipolarization (mP) units, was measured at an excitation wavelength of 485 nm and an emission wavelength of 535 nm, using a DXT 880 plate reader and Multimode analysis software (Beckman Coulter). The ability of test molecules to bind to the PBD was quantitated by a reduction in mP values that occurred as the tracer was displaced from the PBD, and plotted to calculate as an IC₅₀ value.

A similar assay format was developed for the PLK3 PBD to determine the selectivity of PBD inhibitors. The binding of fluorescein-labelled tracer peptides used for both assays was determined to see if there was a difference in affinity for their respective PBD. A titration curve for each tracer/PLK PBD complex was generated to determine Kd values for each and therefore compare the relative binding affinities.

The PLK1 PBD fluorescent tracer was found to bind to the PLK1 PBD with a Kd of 4.6 nM, while the corresponding value for the PLK3 binding tracer to the PLK3 PBD was determined to be 27.2 nM. These results demonstrated that the tracer peptide for the PLK1 PBD bound with a 5-fold higher affinity than the PLK3 tracer to its PBD and this was accounted for when comparing the selectivity of peptides and other drug-like PBD inhibitors described herein. A selectivity index was calculated to provide insights into the true selectivity of compounds, including both previously described compounds and newly generated compounds.

The affinities of the peptides for PLK1 and PLK3 were measured by plotting the loss of polarization against increasing concentration of competitor peptide through use of an optimized assay and selectivity determination. Results are shown in Table 2, below.

TABLE 1

| SEQ ID | | PLK1 PBD FP IC50 | PLK3 PBD FP IC50 | Selectivity |
|---|---|---|---|---|
| NO | Sequence | [μM] | [μM] | Index |
| 1 | LLCS[pT]PNGL | 0.17 ± 0.03 | >600 | >17,000 |
| 2 | LLCSTPNGL | >600 | ND | ND |
| 3 | Ac-PLHS[pT]PNGL | 0.06 ± 0.04 | 163.6 | 13,633 |
| 4 | Ac-PLHS[pT]Al | 0.064 ± 0.01 | 270.5 | 21,133 |
| 5 | Ac-LHS[pT]Al | 1.57 ± 0.47 | >600 | >1900 |
| 6 | Ac-PLHS[pT]A | 0.46 ± 0.12 | >600 | >6500 |
| 7 | GPLATS[pT]PKNG | 0.0057 ± 0.002 | 2.2 | 1928 |
| 8 | GPLATS[pT]PNGL | 0.011 ± 0.003 | 6.3 | 2864 |
| 9 | Ac-PLHS[pT]PKNG | 0.032 ± 0.012 | 133.8 | 20,906 |

The native phospho-sequence from Cdc25C (SEQ ID NO: 1) was determined to potently bind to the PLK1 PBD (IC₅₀=0.17 μM) and to be highly selective with no PLK3 PBD binding apparent at the maximum concentration tested. As previously shown, removal of the phosphate (SEQ ID NO: 2) and introduction of Glu in place of phosphothreonine (results not shown) led to almost complete loss of binding. So as to probe the contributions of the N and C-terminal peptide regions in both the Cdc25C and PBIP context, a series of chimeric peptides were constructed (SEQ ID NO: 3-9). These modifications were found to significantly increase binding compared to either individual context (e.g., SEQ ID NO: 3, IC₅₀=0.06 μM). SEQ ID NO: 3 retains strong selectivity for PLK1 over PLK3 (IC₅₀=163 μM). The two residue sequence (Ala-Ile) C-terminal to the phosphothreonine was found to have comparable contribution to binding as the PNGL C-terminal tetrapeptide (SEQ ID NO: 4, 0.064 μM), while decreasing binding to PLK3 (270 μM), thus improving selectivity. Removal of the N-terminal Pro from this peptide (SEQ ID NO: 5, IC₅₀=1.57 μM) resulted in a large potency drop-off for the PLK1 PBD, which also lost all detectable affinity for the PBD of PLK3. Truncation of the C-terminal isoleucine lead to a 7 fold reduction in binding (SEQ ID NO: 6, IC₅₀=0.46 μM).

SEQ ID NO: 7 is a consensus recognition sequence for PLK3 reported in the literature and was initially synthesized as the fluorescein-labelled tracer for the PLK3 PBD. Testing showed that this peptide (without fluorescein) had a measured IC₅₀ of 2.2 μM in the PLK3 PBD competition assay. However, the somewhat surprising observation was made that this peptide is highly potent for the PLK1 PBD (IC₅₀=0.0057 μM) while retaining almost 2000-fold preferential binding for PLK1 over PLK3.

Two more chimeric molecules were synthesized and tested to examine the consequences of combining the Cdc25C and PLK3 binding motifs. Replacing PKNG of the PLK3 consensus peptide with PNGL from the C-terminus of Cdc25C (SEQ ID NO: 8, IC₅₀=0.011 μM) was found to reduce affinity for both PLK1 and PLK3 by 2 and 3-fold respectively. Furthermore combining the N-terminus of the PBIP sequence with that of the PLK3 consensus sequence (SEQ ID NO: 9, IC₅₀=0.032 μM) modestly reduced affinities for both PBD constructs however, the reduction was more for the PBD of PLK3 thereby significantly improving the selectivity index to 21,000.

Comparison of SEQ ID NO: 1 and SEQ ID NO: 3 revealed that Ac-PLH provides a 3-fold increase relative to LLC and resulted in a highly potent PBD inhibitory peptide with very good PLK1 selectivity. This highlights the contributions of the acetylated N-terminus playing a key role in optimization. Furthermore, a significant observation in comparing the C-terminus of the Cdc25C and PBIP peptides is that the PBD inhibitory activity of the Cdc25C 9mer could be preserved in a 7 residue peptide as evidenced by the similar potency of SEQ ID NO: 4 and SEQ ID NO: 3. This confirms that the Ala-Ile C-terminal dipeptide can provide sufficient affinity to mimic the interactions of the longer sequence. The decrease in size also resulted in increased selectivity of SEQ ID NO: 4 for PLK1 (SI of 21,000) thereby providing the basis for optimization of FLIP compounds utilizing this sequence.

Further truncation of SEQ ID NO: 4 was detrimental to activity in that removal of the isoleucine from the C-terminus led to a 7-fold decrease in PLK1 binding (compare SEQ ID NO: 4 to SEQ ID NO: 6), which suggests an important contribution of this residue in the binding pocket of the PLK1 PBD. Removal of the N-terminal proline from SEQ ID NO: 4 resulted in an even greater loss of binding to PLK1 (˜24-fold compared to 5). These studies strongly indicate that key pharmacophoric elements for potent PLK1 PBD inhibition are contained within the region encompassed by the Ac-PLHS[pT]AI and that these interactions should be preserved in inhibitor optimization and in the search for fragment alternatives for the N and C-terminal determinants.

After it was determined that SEQ ID NO: 7, the consensus recognition sequence for PLK3, was highly potent for PLK1 with single digit nM IC₅₀, the acetylated N-terminal amino acid sequence of PBIP (SEQ ID NO: 6) was combined with the C-terminal PKNG sequence of SEQ ID NO: 7 (forming SEQ ID NO: 9) to probe its relative contribution. Comparative binding data demonstrated that PKNG results in a modestly weakened PLK1 affinity (2-fold) relative to PNGL (compare SEQ ID NO: 9 and SEQ ID NO: 8) while imparting significantly improved selectivity for PLK1. The contributions of PKNG observed by this comparison were further corroborated through relative evaluations with the PNGL C-terminal motif in the PLK3 N-terminal sequence (GPLAT, SEQ ID NO: 7 vs. SEQ ID NO: 8) and a similar 2-fold relative increase was observed.

### EXAMPLE 2

Low molecular weight benzoic acid-based fragments were computationally docked into the volume of a binding site known to interact with key peptidic determinants in order to identify more drug-like alternatives and iteratively convert a peptidic compound into a non-peptidic inhibitor. Through use of the peptide SAR data, fragment alternatives were identified to the N-terminal tripeptide of SEQ ID NO: 1. Its application to the PBD of PLK1 resulted in the identification of peptide-small molecule hybrids that, when transfected into cells, recapitulate a PLK1 deficient phenotype. These FLIPs were based on substituted benzamide capped peptides and demonstrated that modification of the 4 substituent contributed to binding with a hydrophobic slot observed in crystal structures of the PLK1 PBD of PLK1. To extend the structure-activity relationship of this series, capping groups containing additional substituents at the 4-position were incorporated into FLIPs by appending onto the C-terminal portion of the Cdc25C sequence, i.e.,

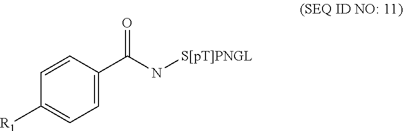

Results are summarized in Table 2.

TABLE 2

| | | PLK1 PBD FP IC50 |
|---|---|---|
| Compound No. | R₁ | [μM] |
| 1 | OH | >600 |
| 2 | OCH₃ | 21.9 ± 4.3 |
| 3 | OCH₂CH₃ | 15.9 ± 2.1 |
| 4 | OCH₂CH₂CH₃ | 10.4 ± 2.6 |
| 5 | OCH₂C₆H₄F | 5.0 ± 0.86 |
| 6 | OCH₂CH₂C₆H₅ | 4.5 ± 0.72 |
| 7 | SCH₃ | 3.0 ± 0.72 |
| 8 | NCH₃ | 11.2 ± 2.6 |
| 9 | NCH₂CH₂CH₃ | 5.4 ± 1.8 |
| 10 | CH₂CH₂CH₂CH₃ | 2.5 ± 0.96 |

In the first instance, the 4-hydroxybenzamide capped FLIP (Compound #1) was completely inactive. The methoxy derivative (Compound #2, IC₅₀=21.9 μM) had measureable inhibition in the PBD FP assay and therefore subsequent homologs were tested in the series. Extending the alkoxy group by 2 (Compound #3) and 3 (Compound #4) carbons resulted in subsequent increases in activity to 15.9 and 10.4 μM, respectively, thereby confirming more effective interaction with the hydrophobic groove and the doubling trend with each successive carbon extension.

Two aromatic substituted alkoxy analogs (Compound #5 IC₅₀=5.0 μM, Compound #6, IC₅₀=4.5 μM) had increased inhibition of the PBD with the 4-phenethoxy (Compound #6) being the slightly more effective of the two. In addition to the methoxy derivative, the thiomethyl and the methylamino FLIP derivatives were synthesized and tested (Compound #7, IC₅₀=3.0 μM, Compound #8, IC₅₀=11.2 μM). The propyl homologue of Compound #8 (Compound #9, 5.4 μM) increased activity by more than 2 fold, consistent with the improvements observed with alkyl chain length in the alkoxy series. In addition, these were compared with the 4-butyl benzamide FLIP previously tested (Compound #10, IC₅₀=2.5 μM) and revealed that the thiomethyl analog (Compound #7) is of comparable potency to the longer alkyl chain (Compound #10). It was therefore apparent in comparison of this isosteric series that the order of S>C>N>O was observed in terms of potency of inhibition of the PBD. Further validation for extension of the alkyl portion of the substituent as a means of improving potency was confirmed by comparison of the propylamino (Compound #9) with Compound #10 and the resulting two-fold potency increase.

As the above results reveal that the ethyl substituent increased potency in this isosteric series, further homologation of the alkyl series was the obvious next step in the SAR. A FLIP series generated by lengthening the 4-alkyl substituent were then tested in the FP assay. Compounds 11-16 included the R₁ group on the N-terminal of the identified sequence. Compounds 17 and 18 included the R₁ group on the C-terminal of the identified sequence. Results are provided in Table 3, below.

TABLE 3

| Compound | | | PLK1 PBD FP | PLK3 PBD FP | Selectivity |
|---|---|---|---|---|---|
| No. | R₁ | Sequence | IC50 [μM] | IC50 [μM] | Index |
| 11 | 4-butyl | S[pT]PNGL | 2.5 ± 0.96 | >600 | >1200 |
| | | (SEQ ID NO: 11) | | | |
| 12 | 4-hexyl | S[pT]PNGL | 1.0 ± 0.51 | >600 | >2830 |
| | | (SEQ ID NO: 11) | | | |
| 13 | 4-octyl | S[pT]PNGL | 0.36 ± 0.16 | 148.8 ± 38.2 | 2067 |
| | | (SEQ ID NO: 11) | | | |
| 14 | 4-octyl | S[pT]A | 15.2 ± 4.74 | 201.9 ± 51.3 | 66 |
| | | (SEQ ID NO: 12) | | | |
| 15 | 4-octyl | S[pT]Al | 0.41 ± 0.14 | 152.8 ± 33.5 | 1863 |
| | | (SEQ ID NO: 13) | | | |
| 16 | 4-octyl | S[pT]PL | 1.2 ± 0.18 | 620.1 ± 72.5 | 2583 |
| | | (SEQ ID NO: 14) | | | |
| 17 | 4-octyl | S[pT]Al | 1.49 ± 0.13 | 116.7 | 79 |
| | | (SEQ ID NO: 13) | | | |
| 18 | 4-nonyl | S[pT]Al | 1.23 ± 0.81 | 95.87 | 78 |
| | | (SEQ ID NO: 13) | | | |

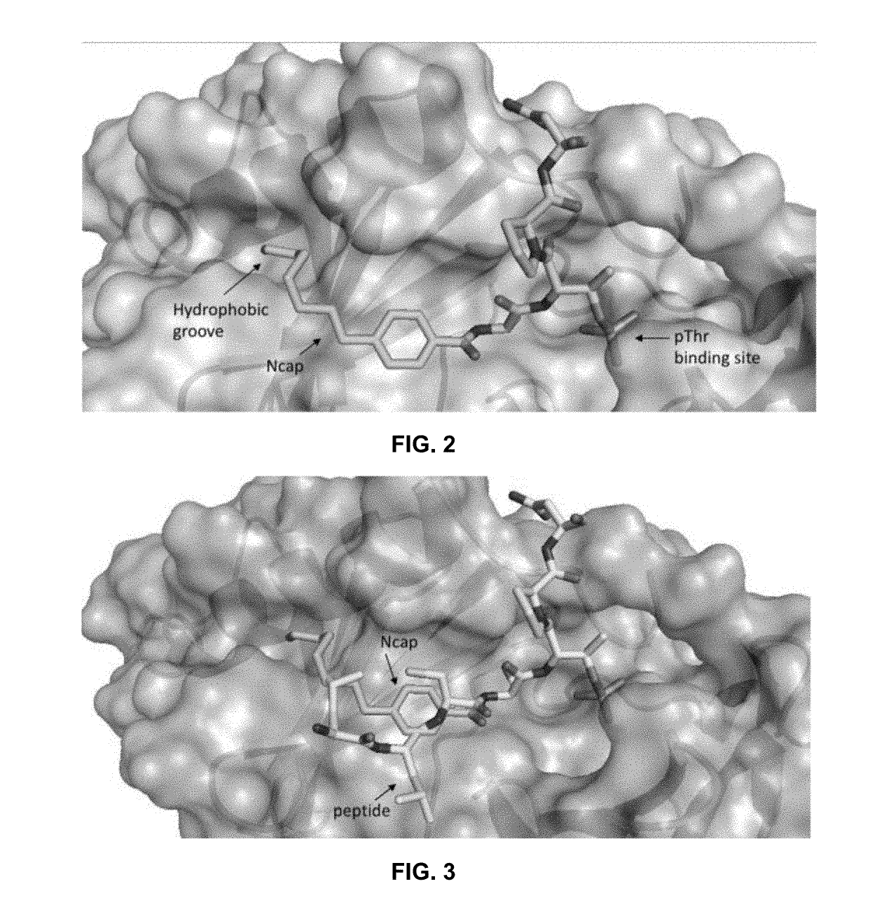

Compound No. 11 (butyl, IC₅₀=2.5 μM), Compound No. 12 (hexyl, IC₅₀=1.0 μM), and Compound No. 13 (octyl, IC₅₀=0.36 μM) overall demonstrated significantly improved affinity for the PLK1 PBD with increasing length of the alkyl substituent. Notably, the octyl derivative (Compound No. 13) was found to essentially recapitulate the activity of the native Cdc25C peptide (SEQ ID NO: 1). Although Compound No. 13 measurably bound to the PBD of PLK3 (IC₅₀=148 μM), it possesses a 2000-fold selectivity index and thereby retains high specificity for PLK1. FIG. 2 and FIG. 3 illustrate interactions of the alkyl group of the benzamide capping group of Compound No. 13 with the PBD of PLK1 (PBD ID: 3RQ7). The hydrophobic groove exploited by the capping group and the interactions of the phosphothreonine are indicated. In FIG. 3, the original peptide group is overlaid with the replacement benzamide capping group of Compound No. 13.

Further C-terminal modifications were explored in the context of the octyl-benzamide fragment alternatives (Table 3, Coompound No. 17 and 18).

Since the peptide SAR results with SEQ ID NO: 4 (Table 1, 0.064 μM) showed that the PNGL sequence could be replaced with two residues without significant potency loss, a 4-octylbenzamide N-capped S[pT]AI (SEQ ID NO: 13) compound (Compound No. 15) was constructed. As expected this molecule (IC₅₀=0.41 μM) possessed very similar activity to Compound No. 13 while retaining its selectivity (SI=2000). Additionally, the consequences of further truncation were determined by deleting the C-terminal Ile residue. The resulting FLIP (Compound No. 14, IC₅₀=15.2 μM) containing a single alanine C-terminal to the phospho-Thr bound to the PLK1 PBD with a ˜40-fold weaker affinity revealing a substantial potency loss, which was greater than expected based on the peptide SAR. The binding of Compound No. 14 to PLK3 was measurable (IC₅₀=201 μM) and therefore had dramatically reduced selectivity compared to Compound No. 13 (SI of 66). When the C-terminal Al of Compound No. 15 was replaced with proline and leucine, the resulting FLIP (Compound No. 16) bound to both PLK PBDs with 3-4 fold weaker affinity and therefore retained selectivity for PLK1.

These results clearly demonstrate the optimization of the capping group through extension of the alkyl chain to 6 carbons (Compound No. 12) and that the potency of the native Cdc25C peptide (SEQ ID NO: 1) can be recapitulated in the FLIP molecule with sub-micromolar activity by incorporating a 4-octyl substituent (Compound No. 13). Molecular modeling showed that linear alkyl chain exploits a hydrophobic groove and that extension of this chain interacts to a greater extent (FIG. 2). While this groove has been exploited in previously described compounds, the 4-alkylbenzamide structure represents a low MW and simple strategy to obtain highly potent but more drug-like PBD inhibitors. Furthermore, the peptide SAR knowledge was applied in the FLIP context to generate capped peptides with only 4 residues (Compound No. 15) and which preserve the activity of the 6mers ligated to the benzamide groups (Compound No. 13 vs. Compound No. 15) while maintaining high levels of selectivity for PLK1 vs PLK3 (SI=>1800) and confirmed that the C-terminal Ile is critical for potent FLIP activity.

### EXAMPLE 3

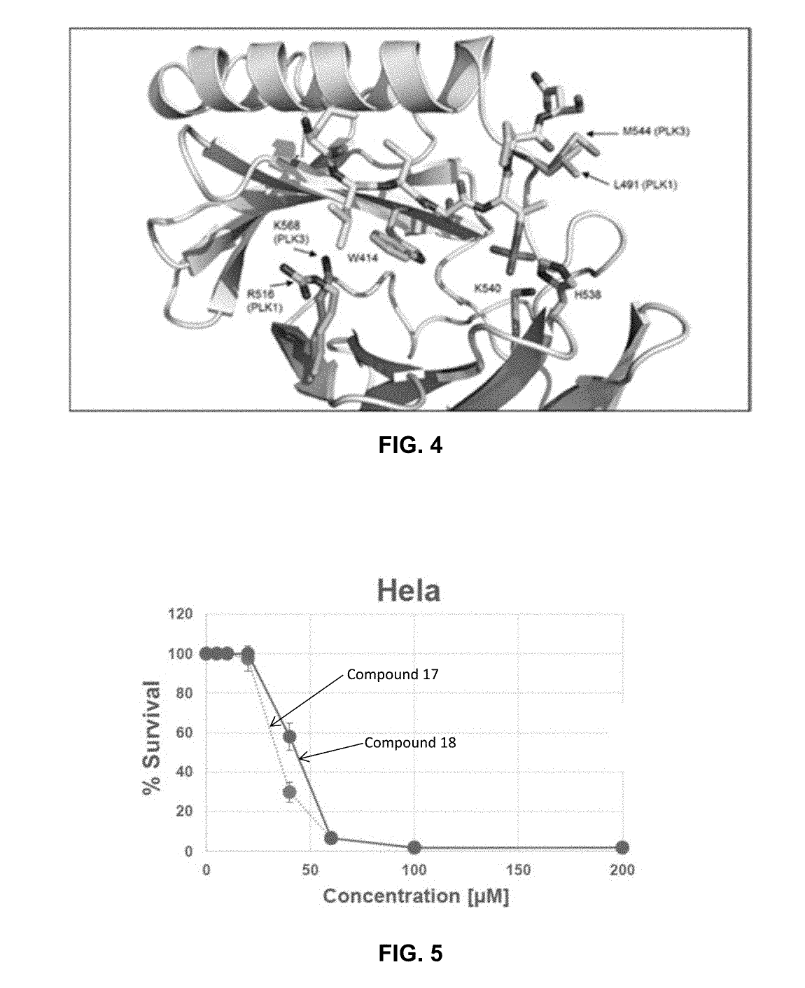

In order to shed light into the structural basis for the mimicry of the capping groups and to determine why peptides and FLIPS are selective for PLK1, a homology model for the PBD domain of PLK3 was constructed and compared with crystal structures for the PBD of PLK1. The PLK3 PBD model was then overlaid with the crystal structure of the cdc25c peptide (SEQ ID NO: 1) used as a starting peptide. The resulting model is illustrated in FIG. 4.

A striking observation from the sequence and structural alignment was that the majority of residues involved in the phosphopeptide binding interface are strictly conserved between the two homologs and therefore offer little insight into the differential binding of PBD inhibitors. This led to the conclusion that the dramatically weaker binding of the phosphopeptides largely results from conformational differences between the PBD domains of PLK1 and PLK3. Closer examination of the minor sequence differences however did provide insights into possible reasons for PLK1 selectivity. One of these variations is L491 of PLK1, a residue that directly contacts the phosphothreonine residue in the PBD crystal structure. In the PLK3 PBD, the corresponding residue is M544 and based on the modelling results does not appear to make contacts with the methyl group of the pThr residue that are observed with L491 in PLK1. It is possible that L491 acts as an anchor for the phosphothreonine (critical for high affinity binding) and therefore acts to solidify the many other contacts of this residue. The absence of these contacts could thus result in dramatically lower binding affinity in the PLK3 PBD context.

### EXAMPLE 4

With the increased activity in binding to the PBD, and as the FLIPs described here have greater log P values, e.g., through addition of the 4-octylbenzamide capping group and decreased overall size (after deletion of two C-terminal residues), it was hypothesized that these FLIPs may possess drug-like properties and cellular activity without the need of a delivery agent. Accordingly, cell viability was measured using an MTT assay to determine the anti-proliferative activity for Compounds No. 15, 16, 17, and 18 with regard to three different cell types (HeLa, PC-3, and A549). The cells utilized were chosen due to reported synthetic lethal interactions with PLK1 inhibition

Results are shown in Table 4, below.

TABLE 4

| | MTT IC50 [μM] | MTT IC50 [μM] | MTT IC50 [μM] |
|---|---|---|---|
| Compound No. | HeLa | PC-3 | A549 |
| 15 | 128.1 | 55.7 ± 2.7 | 79.45 ± 16.6 |
| 16 | 142.3 ± 14.4 | 102.7 ± 27.3 | 160.5 ± 61 |
| 17 | 38.9 ± 0.6 | 41.5 ± 6.5 | |
| 18 | 38.2 ± 11.7 | 27.0 ± 6.4 | |

Despite still having a peptidic composition and a negatively charged phosphothreonine, these FLIPs demonstrated cellular activity. Moreover, these compounds displayed a measurable antiproliferative effect without additional modification (e.g., PEGylation, histidine derivatization, and/or masking of the phosphothreonine) or the use of or a drug delivery agent.

Compound No. 15 had anti-proliferative IC₅₀ values in PTEN deficient prostate cancer (PC-3) cells of 55.7 μM, in HeLa cells of 128.1 μM, and Kras mutant (A-549) lung cancers cells of 79.5 μM. The anti-proliferative activity of Compound No. 16 in the same cells was approximately 2-fold lower yet clearly measurable and consistent with its decreased binding to PLK1 PBD.

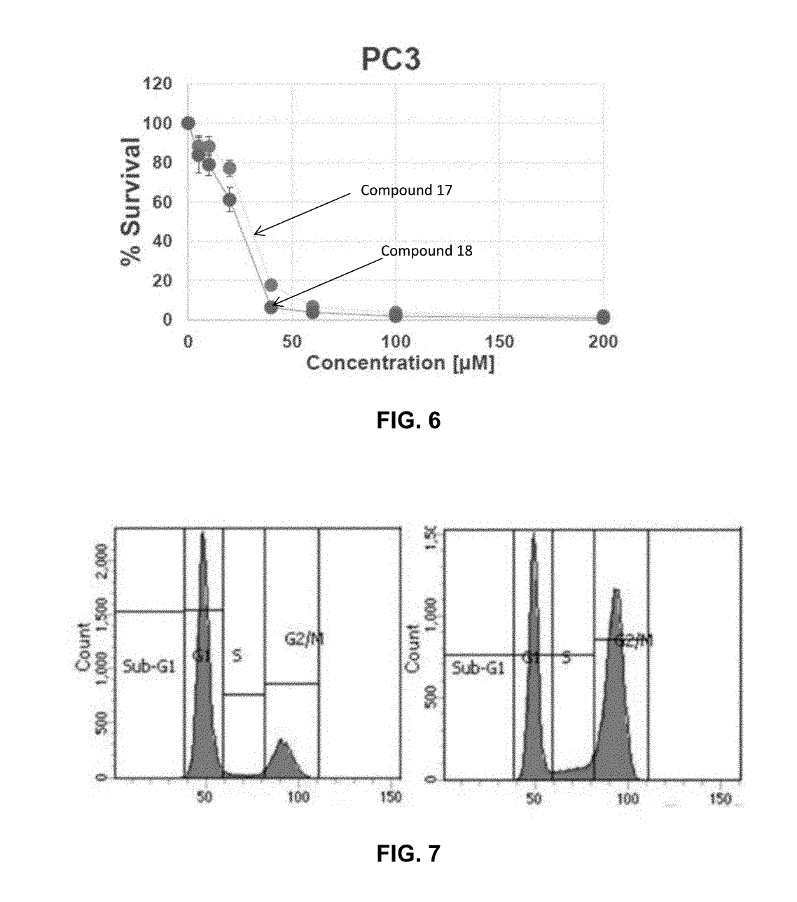

FIG. 5 and FIG. 6 graphically illustrate the anti-proliferative activity of Compound Nos. 17 and 18 in HeLa (FIG. 5) and PC3 (FIG. 6) cells. The neutral C-terminal amidated octyl and nonyl FLIPs (Compounds 17 and 18) showed significantly higher anti-proliferative activity than the N-terminated octyl FLIPS.

To examine the cellular phenotype of PLK1 inhibition through the Polo-box domain, PC3 cells synchronized in G1 through serum starvation were treated with Compound No. 16. Cells were synchronized by serum starvation for 24 hours prior to treatment. Results are shown in FIG. 7. Untreated cells were serum starved and then released into drug-free media for 24 hours (left panel) as control. Sample cells were serum starved and then treated with 200 μM Compound No. 16 for 24 hours (right panel). The results demonstrated that a significant accumulation in G2/M was observed (53% compared to 19.9% for mock treated cells and therefore was consistent with the depletion of PLK1.

While no strict conclusions can be made, the anti-proliferative activity of the tested cells was nonetheless consistent with ability of the compounds to bind to the PBD. Furthermore, cell cycle distribution experiments demonstrated that the optimized FLIPs induce a presumptive mitotic arrest that phenocopies the mode of action of B12536 and other methods of down regulating PLK1 activity.

As it was shown that optimized FLIPs had cellular activity resulting from nascent drug-like properties, potential application of these for tumors that have developed resistance to clinically utilized ATP competitive inhibitors of PLK1 was explored. Retinal pigment epithelial (RPE) cells, which have previously been utilized to demonstrate that a single point mutation (C67V) within the ATP-binding domain of PLK1 confers resistance to several structurally unrelated ATP-binding site inhibitors, including BI-2536, were obtained and used to test FLIPs in this model of resistance to catalytic inhibitors of PLK1. The RPE cells expressing wild-type PLK1 were very sensitive to BI-2536 (IC₅₀=21.2 nM) while those expressing the C67V mutant are dramatically resistant to this compound (>2.5 μM, results not shown)

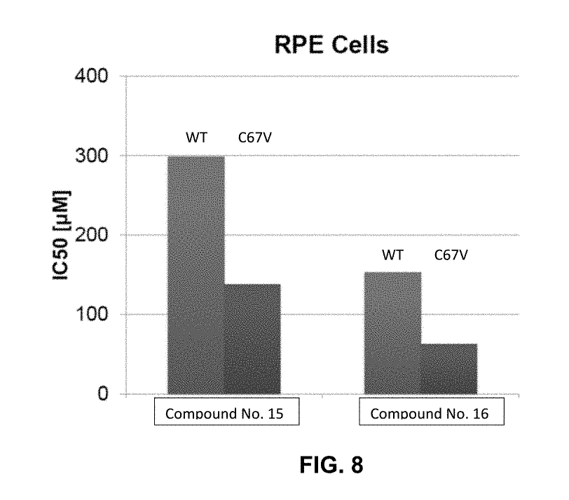

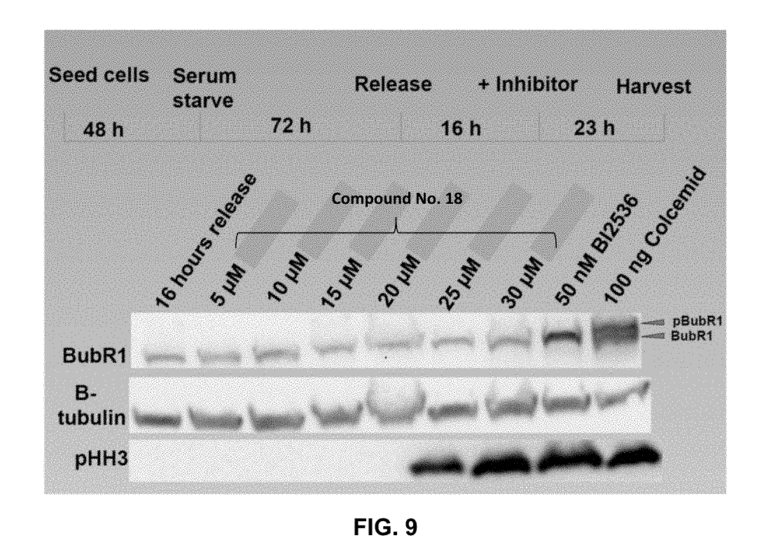

Results of these cells with regard to Compound Nos. 15 and 16 are shown in FIG. 8. As shown, the PBD-targeted Compound No. 16 inhibited the growth of the PLK1 C67V mutant-expressing cells to an equal or even greater extent as that seen with cells expressing wild-type PLK1 (86.8 μM±33.8 versus 158.5 μM±8.8, respectively). These cells were sensitive to both FLIPs, however, thereby demonstrating that blocking the PBD could be a synergistic approach with ATP blockers currently in clinical development.

To measure the PLK1 inhibitory activity of the FLIPs in cells, phosphorylation of the mitotic protein BubR₁ was measured. PC-3 cells were serum starved for 72 h and released from the G1-S boundary. 16 h after released, cells were arrested at prometaphase by treatment with 100 ng/ml Colcemid or treated with 50 nM BI2536 PLK1 kinase inhibitor or 5-30 μM of Compound No. 18 for 23 hours. Cells were trypsinized and whole cell lysate was prepared. The obtained total cell lysate was subjected to immunoblotting with anti-BubR₁ (top panel, FIG. 9), anti-β tubulin (middle panel, FIG. 9), and anti-pHH3 (bottom panel, FIG. 9). As shown, Compound No. 18 blocked the cells at mitosis and inhibited the BubR₁ phosphorylation. Overall these results highlight that targeting the PBD of PLK1 has promise as an antitumor strategy.

It will be appreciated that the foregoing examples, given for purposes of illustration, are not to be construed as limiting the scope of this disclosure. Although only a few exemplary embodiments have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure which is defined in the following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the present disclosure.

What is claimed is:

1. An inhibitor that targets a polo-box domain of a polo-like kinase 1, the inhibitor comprising a benzoic acid-based fragment having the following structure: 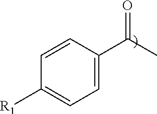
wherein R₁ comprises an alkyl group, an alkoxy group, an alkylthio group, an alkylamino group, or a phenyl alkoxy group.

2. The inhibitor of claim 1, wherein the benzoic acid-based fragment is bonded to a peptide fragment.

3. The inhibitor of claim 2, the peptide fragment comprising SEQ ID NO: 11, SEQ ID NO: 12, SEQ ID NO: 13, or SEQ ID NO: 14.

4. The inhibitor of claim 2, wherein the benzoic acid-based fragment is bonded to the peptide fragment at the N-terminal of the peptide fragment.

5. The inhibitor of claim 2, wherein the benzoic acid based fragment is bonded to the peptide fragment at the C-terminal of the peptide fragment.

6. The inhibitor of claim 1, wherein R₁ comprises a C6 or longer alkyl chain.

7. The inhibitor of claim 1, wherein R₁ comprises a phenyl alkoxy group.

8. The inhibitor of claim 7, the phenyl of the phenyl alkoxy group further comprising a derivatization.

9. The inhibitor of claim 8, the derivatization of the phenyl comprising a halogen.

10. A method for inhibiting the proliferation of a cell population, the cell population comprising cells that express polo-like kinase 1, the method comprising contacting the cell population with an inhibitor, the inhibitor comprising a benzoic acid-based fragment having the following structure: 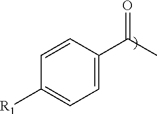
wherein R₁ comprises an alkyl group, an alkoxy group, an alkylthio group, an alkylamino group, or a phenyl alkoxy group.

11. The method of claim 10, cells of the cell population expressing polo-like kinase 3, wherein the inhibitor exhibits a selectivity index for polo-like kinase 1 over polo-like kinase 3 of about 100 or more.

12. The method of claim 10, wherein the inhibitor is a fragment ligated inhibitory peptide.

13. The method of claim 10, wherein the inhibitor is a non-peptidic inhibitor.

14. The method of claim 10, wherein the cells that express polo-like kinase 1 comprise cancer cells.

15. The method of claim 14, wherein the cancer cells comprise prostate cancer cells or lung cancer cells.

16. The method of claim 10, wherein the inhibitor is free of PEGylation, histidine derivation and phosphothreonine masking.

17. The method of claim 10, wherein the cell population is resistant to an ATP competitive inhibitor.

18. The method of claim 16, wherein the ATP competitive inhibitor comprises BI2536 or BI6727.
